(19) United States
(12) Patent Application Publication
(10) Pub. No.: US 2003/0099930 A1
(43) Pub. Date: May 29, 2003

(54) Novel method to enhance microarray surface density and hybridization efficiency

(76) Inventors: David J. Graves, Devon, PA (US); David Cossaboon, Germantown, MD (US); Richard Hung Ju Su, Arcadia, CA (US); Paolo Fortina, Philadelphia, PA (US); Saul Surrey, Chadds Ford, PA (US); Steve McKenzie, Springfield, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/052,972

(22) Filed: Oct. 29, 2001

Related U.S. Application Data
(60) Provisional application No. 60/244,110, filed on Oct. 27, 2000.

(51) Int. Cl. C12Q 1/70; C12Q 1/68; G01N 33/53; G01N 33/567; C12M 1/34; B05D 3/00

(52) U.S. Cl. 435/5; 435/6; 435/7.2; 427/2.11; 435/287.2

(57) ABSTRACT

Disclosed are nucleic acid microarray devices and their method of preparation employing dendrimeric polyamines to bind nucleic acid probes to solid support surfaces thereby enhancing the surface molecule density and hybridization efficiency of nucleic acids in the microarray. Also disclosed are methods of using the resulting microarray device for analyzing test samples containing target molecules.

This application claims the benefit of U.S. Provisional Application No. 60/244,110 filed Oct. 27, 2000, the entire disclosure of which is incorporated by reference herein.

### FIELD OF THE INVENTION

This invention relates generally to microarray technology, and more specifically to methods of producing microarrays of biological entities, preferable nucleic acids, that exhibit enhanced surface molecule density and efficiency of interactions with binding partners.

### BACKGROUND OF THE INVENTION

DNA microarray technology has emerged as a powerful tool for discovering genetic information. The application of this revolutionary technology, embodied in what are known as DNA chips, has resulted in explosive discoveries in the fields of health-related sciences and medicine. The major applications of DNA microarrays are divided in the two categories: studies of genomic structure and studies of active gene expression. The former includes genetic disease diagnosis (e.g., mutation detection), polymorphism analysis (e.g., SNP analysis), gene mapping, and sequencing by hybridization. The latter mainly provides information about which genes are currently active in a given sample and at what level. Such information aids in understanding the phenotype of an organism, which determines its form and function.

In its most basic form, a DNA microarray is simply a solid support, e.g. glass or silicon, bearing on its surface an array of different DNA fragments (called “probes”), usually having a known sequence, at discrete locations or spots on the support. The DNA spots on the chip are hybridized to detectably labeled nucleic acid molecules (called “targets”) which are present in a test sample. The pattern and extent of detectable label, e.g. fluorescence, that is observed provides information about the nucleic acids present in the solution, either qualitatively in searching for the presence of a particular sequence (for example, mutation detection), or quantitatively, in attempting to determine the amount of numerous sequences likely to be present (as in gene expression patterns).

Microhybridization arrays on glass slides enable heterogeneous hybridization between the target nucleic acids and the probes. Each microarray consists of several hundred to several hundred thousand microscopic spots. Each spot in the array contains single strand oligonucleotide probes which are usually 10-30 bases long or complementary DNA (cDNA) probes, typically 500-1,000 bases long. The amount of the probe attached to the solid support is small and the spots are closely spaced. Thus, the consumption of probe solution to make spots and the volume of target-containing test solution are both low. The probes are conventionally attached to the solid support by chemical linkage or physical adsorption. A solution phase of oligonucleotides or single-stranded DNA labeled with a detectable reporter is then contacted with the support surface. Only two complementary strands, one in the liquid phase and the other on the solid phase, will hybridize under appropriate conditions of hybridization and washing. The support is then brought to a suitable detection instrument to determine the degree of hybridization.

There are at least three general methods of fabricating microarrays. The first, pioneered by Affymetrix Corporation, involves using microlithographic techniques similar to those used to manufacture integrated circuits. This method can produce arrays with half a million sequences on a surface, but has two major drawbacks. The masks used cost tens of thousands of dollars, and they are totally inflexible. Once a mask has been designed, one cannot change a sequence or add a new one to the array without repeating the expense. Furthermore, synthesizing the probes (typically 20 bases long) directly on the surface is error prone and the quality of each spot is rather poor. Typically, there must be 10 or 20 different spots to be sure of a particular result.

The second method for producing microarrays involves using relatively long cDNA's as the probe molecules. If the sequences are long enough (typically over 300 bases) they will adsorb irreversibly on the surface of the solid support. A robotic spotter is used to deposit microdroplets of a DNA on the surface to make the arrays.

The third method uses relatively short oligonucleotides (typically 20 bases) that are spotted using a robotic spotter and the oligonucleotides attach to the solid support via chemical linkage. This method is attractive because of its flexibility, low cost and simplicity.

An additional method of preparing microarrays involves the synthesis of a dendrimeric linker system on a glass surface which facilitates the immobilization of nucleic acid compounds (Beier, M., and Hoheisel, J. D., Nucleic Acid Research, 27, 1970-1977 (1999)). The major drawback to this method is that it requires a long series of tedious organic chemical reactions to create the dendrimeric linker structure on the glass surface.

A major drawback to nearly all microarrays is that the common substrates employed (i.e., glass or silicon) do not provide a sufficiently dense surface coverage of molecules. To overcome this limitation, expensive scanners ($100,000 or more) are required to read out the results of a hybridization experiment. Microarrays fabricated using various plastic substrates can achieve about a 10-fold improvement in density; however, glass is still the preferred substrate material because of its low intrinsic fluorescence, availability of microscope slides at low cost and ease in handling and archiving.

Another drawback to the existing methods of microarray production is that in order to obtain high yields of attached oligonucleotides, aliphatic amines must be added to the oligonucleotides during their synthesis in order to covalently attach the oligonucleotide probes to the solid support surface. Additionally, the results are much improved if the amines are placed at the end of a relatively long chain or “spacer” molecule (typically 18 to 54 carbon bonds in length) that is attached directly to the oligonucleotide to prevent stearic hindrance from decreasing the hybridization efficiency. Although ordinary oligonucleotides can be purchased at relatively low cost (approximately $20 each for enough material to make thousands of microarrays), the addition of the reactive amine doubles the price, and adding an amine plus a spacer molecule can increase the cost by 10-fold. If the end use requires an array with thousands of sequences, the cost to produce the necessary oligonucleotide probes will be extraordinarily high.

### SUMMARY OF THE INVENTION

In accordance with the present invention, a novel method has been devised which increases the density of biological entities on a solid support surface to improve efficiency of interactions with specific binding partners and purification and separation processes.

The method of the invention involves preparing microarrays of biological entities having at least one functional amine reactive group by first treating a solid support, e.g., a glass slide, with a dendrimeric polyamine such that the dendrimeric polyamine is affixed to a surface of the support. A biological entity-containing solution is then microspotted on the surface of the dendrimeric polyamine-bearing support under conditions which cause the chemical binding of biological entities to the amine groups of the dendrimeric polyamine. In a preferred embodiment of the invention, nucleic acid molecules are microspotted on a surface of the dendrimeric polyamine-bearing support to produce nucleic acid microarrays. In addition, biological entities including proteins, phages, eucaryotic cells, procaryotic cells and viruses are microspotted on a surface of the dendrimeric polyamine-bearing support to enhance purification and separation processes.

Nucleic acid microarrays fabricated using this method comprise a suitable substrate glass with a surface bearing dendrimeric polyamine, and a plurality of different nucleic acid probes chemically bound to the dendrimeric polyamine, the latter forming a pattern of discrete microspots on the glass slide, with each microspot being composed of a different nucleic acid probe.

In a particularly preferred embodiment of the invention, polyamidoamine (PAMAM) dendrimers are affixed to the surface of a glass slide by physical adsorption. After affixing the PAMAM dendrimers to the surface of a glass slide, the slides are preferably stored for a period of at least two weeks before microspotting the biological entitye-containing solution onto the PAMAM dendrimer-bearing glass slide. The PAMAM dendrimers may also be affixed to the surface of a glass slide by covalent bonding.

In other embodiments of the invention, dendrimeric polyamines including symmetrical and unsymmetrical branching dendrimers, lysine-based dendrimers and nucleic acid dendrimers may be affixed to the surface of a glass slide.

In yet another aspect of the invention there is provided a method for analyzing a test sample for the presence of at least one target nucleic acid molecule using the above-described dendrimeric polyamine-bearing microarrays. This method comprises the steps of: (a) providing a nucleic acid microarray as described above, (b) contacting the nucleic acid microarray with a test sample under conditions causing the formation of hybrids between the target nucleic acid molecules in the test sample and the nucleic acid probes microspotted on the nucleic acid microarray, (c) labeling with a detectable reporter substance either the test sample containing the target nucleic acid molecules or the hybrids formed between the target molecules and the nucleic acid probes, and (d) detecting the occurrence of the detectable reporter substance on the nucleic acid microarray. In a preferred embodiment, the target molecules are labeled with a fluorescent dye, such as Cy3 or Cy5.

### BRIEF DESCRIPTION OF THE DRAWINGS

FIG. 1 is an illustration of the amino-terminal branching of a G2 polyamidoamine dendrimer.

FIG. 2 shows images obtained with a scanning confocal microscope system of two DNA microarray slides, one slide treated with dendrimer (right) and the other slide without dendrimer (left). The attachment control probe, FC3, was spotted in 16 duplicates on both slides.

FIG. 3 is a graphical illustration of the signal intensities measured from the microarrays in FIG. 2.

FIG. 4 shows images obtained with a scanning confocal microscope system of two DNA microarray slides, one slide treated with dendrimer (right) and the other slide without dendrimer (left). The top two rows on each slide were spotted with the non-spacer probe, FC2, and the bottom two rows were spotted with the spacer probe, FC2SP.

FIG. 5 is a graphical illustration of the signal intensities measured from the microarrays in FIG. 4.

FIG. 6 shows an image obtained with a scanning confocal microscope system of a DNA microarray slide that was coated with 0.1 wt % polyamidoamine dendrimer solution. The top row was spotted with a mixture of the 600 bp oligonucleotide probe and ArrayIt® solution and the middle row was spotted with a mixture of the 300 bp oligonucleotide probe and ArrayIt® solution. The bottom row was spotted with the 300 bp oligonucleotide probe without ArrayIt® solution.

FIG. 7A is a graph of the DNA signal intensities from G2 and G4 polyamidoamine dendrimers as a function of dendrimer concentration. FIG. 7B is a graph of the signal to noise (S/N) ratios of G2 and G4 polyamidoamine dendrimers as a function of dendrimer concentration.

FIGS. 8A-8C show images obtained with a scanning confocal microscope system of three nucleic acid microarray slides bearing polyamidoamine dendrimers that were spotted with a solution containing a 600 bp oligonucleotide probe after one day (FIG. 8A), one week (FIG. 8B) and two weeks (FIG. 8C).

### DETAILED DESCRIPTION OF THE INVENTION

The invention will be described hereinbelow with particular emphasis on the production and use of dendrimeric polyamine-bearing nucleic acid microarrays. It should be understood, however, that the invention has a substantially broader range of utilities involving the affixation of a wide range of biological entities to solid supports, including but not limited to, proteins, phages, eucaryotic cells, procaryotic cells and viruses, to enhance purification and separation processes.

In order to improve signal detection on DNA microarrays and to eliminate the high cost of synthesizing oligonucleotides with both reactive amine groups and spacer molecules, a novel yet inexpensive method to produce DNA microarrays with enhanced surface molecule density and hybridization efficiency has been developed. In carrying out the method of the invention, a preformed multivalent molecule is attached to the surface of the solid support rather than directly to an oligonucleotide probe to increase the density of the oligonucleotide probes affixed to the solid support surface and to enhance the hybridization efficiency on each array.

In a particularly preferred embodiment of the present invention, polyamidoamine (PAMAM) dendrimer molecules, also known as Starburst® dendrimers (a trademark of Dendritech, Inc.), are affixed to a solid support surface to create dendrimeric polyamine-bearing substrates for nucleic acid microarrays. The dendrimeric polyamine-bearing substrates of the invention may be used to advantage to perform routine microarray analyses, whereby probes that are capable of binding to target molecules are bound onto the dendrimeric polyamine-bearing substrates in discrete microspots to complete the formation of the nucleic acid microarray.

The use of dendrimers on nucleic acid microarray supports is advantageous for microarray hybridization reactions because: (1) purchasing and attaching dendrimers to the surface of solid supports is more economical than purchasing oligonucleotides modified with amine groups, with or without spacer molecules; (2) the dendrimers enhance the density of the nucleic acid molecules that can be bound to the supports; and (3) the number of reactive amine groups may be altered by choosing dendrimers of different configurations to eliminate stearic hindrance on the surface of the glass slide which will promote greater hybridization efficiency.

I. Definitions

The following definitions are provided to facilitate an understanding of the present invention:

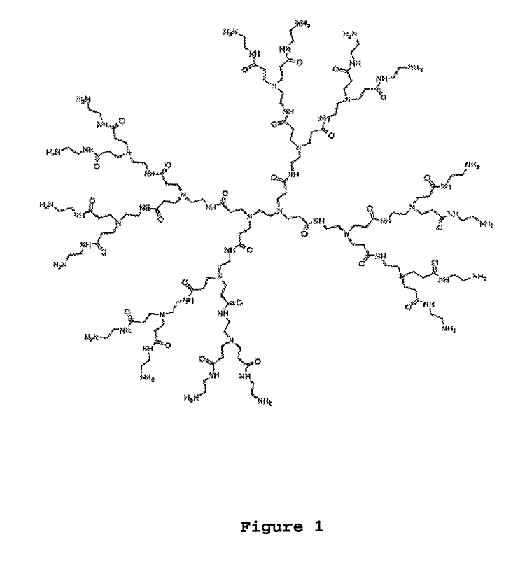

“Dendrimeric polyamines” as used herein refers to molecular constructs in which branch-like structures having amine terminal groups extend radially from a core moiety. The arms may be linear or branched and may further comprise closed branched structures such as loops. Dendrimeric polyamines include but are not limited to, symmetrical and unsymmetrical branching dendrimers, cascade molecules, arborols, and the like, though the most preferred dendrimeric polyamines are polyamidoamines or PAMAM dendrimers. The PAMAM dendrimers are generally symmetric, in that the branched arms are of equal length, and the branching occurs at the hydrogen atoms of a terminal amine group on a preceding generation branch. PAMAM dendrimers may be purchased with 2, 4, 8, 16, 32 etc. (up to 4096) reactive amine groups at the end of the branched arms which emanate from a common ethylenediamine core moiety. The dendrimers are designated G0, G1, G2, G3 etc. depending on the number of sequentially branched layers or generations that are used to form them. For example, a G2 dendrimer comprises 16 reactive amino groups (see FIG. 1).

The expression “amine reactive functional groups” as used herein with reference to any of the above-mentioned biological entities, signifies a chemical functionality that is capable of undergoing a chemical reaction with a free amine group, such as those present on the dendrimer.

With reference to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. An “isolated nucleic acid molecule” may also comprise a cDNA molecule or a recombinant nucleic acid molecule.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The term “oligonucleotide,” as used herein refers to sequences and probes of the present invention, and is defined as a nucleic acid molecule comprised of two or more ribo- or deoxyribonucleotides, preferably more than three. The exact size of the oligonucleotide will depend on various factors and on the particular application or end use of the oligonucleotide.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989)):

Tₘ=81.5° C.+16.6 Log[Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. In certain applications, such as mutation detection, it is important to be able to distinguish targets with perfect complementarity from those with a single base mismatch. Such stringency is possible under the proper conditions.

The term “probe” as used herein refers to an oligonucleotide, polynucleotide or DNA molecule, whether occurring naturally as in a purified restriction enzyme digest or produced synthetically, which is capable of annealing with or specifically hybridizing to a nucleic acid with sequences complementary to the probe. More particularly, the probes described herein are the oligonucleotides attached to a solid support in making the DNA microarray device of the invention. A probe may be either single-stranded or double-stranded. The exact length of the probe will depend upon many factors, including temperature, source of probe and use of the method. For example, for diagnostic applications, depending on the complexity of the target nucleic acid sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin, amine-reactive agent-amine conjugated molecule and thiol-gold interactions. Various other determinant-specific binding substance combinations are contemplated for use in practicing the methods of this invention, such as will be apparent to those skilled in the art.

The term “detectable label” is used herein to refer to any reporter substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

II. Application of Dendrimeric Polyamines to Microarrays:

In a particularly preferred embodiment of the invention, G2 polyamidoamine (PAMAM) dendrimers with 16 amino groups are affixed to a solid support surface. Most preferably, the solid support is a glass microscope slide; however, the use of other solid supports such as plastic substrates or nylon membranes is also contemplated to be within the scope of the invention. In addition, PAMAM dendrimers containing 4, 8, 32 and 64 reactive amino groups, respectively, may be utilized in practicing the present invention.

The preferred method of affixing PAMAM dendrimers to the surface of a solid support is by physical adsorption. In addition, PAMAM dendrimers may be affixed to the surface of a solid support by covalent linkage. For example, PAMAM dendrimers may be affixed to a solid support by first treating the solid support surface with 3-aminopropyltriethoxysilane (APTES) and 1,4 phenylene diisothiocyanate (PDC) followed by the addition of PAMAM dendrimers. APTES will coat the surface of the solid support and PDC will attach to the APTES coating and covalently link the PAMAM dendrimers to the surface of the solid support. This chemical linkage may be used to advantage to create other forms of microarrays, such as protein arrays, whereby an additional layer of PDC is attached to the dendrimers covalently linked to the surface of the solid support which can bind non-nucleic acid target molecules such as proteins.

In another embodiment of the invention, covalent linkages may also be formed between the PAMAM dendrimers and the surface of the solid support by applying polylysine to the surface of the solid support. Polylysine is positively charged and will bind to surfaces that have a net negative charge, such as glass.

In yet another embodiment of the invention, the dendrimeric polyamines are affixed to the surface of the solid support such that the entire surface area is covered with dendrimeric polyamines. The uniform distribution of dendrimeric polyamines across the entire surface area enhances the density of probes microspotted onto the surface of the solid support. In addition, the dendrimeric polyamines may be affixed to the surface of the solid support in any preferred geometric configuration or pattern. Affixing the dendrimeric polyamines to the surface of the solid support in this manner may be utilized to suppress background signal by preventing indiscriminate sticking of the dendrimeric polyamines to the surface of the solid support.

In a related aspect of the invention, other forms of dendrimeric polyamines may be affixed to the surface of the solid support. Such dendrimeric polyamines include, but are not limited to, symmetrical and unsymmetrical branching dendrimers, lysine-based dendrimers and nucleic acid dendrimers. “Lysine-based dendrimers” as used herein are unsymmetric, in that the branched arms are of a different length. One branch occurs at the epsilon nitrogen of the lysine molecule, while another branch occurs at the alpha nitrogen, adjacent to the reactive carboxy group which attaches the branch to a previous generation branch. “Nucleic acid dendrimers” as used herein are composed of individual molecules consisting of two DNA strands that share a region of sequence complementarity located in the central portion of each strand. When the two strands anneal together, the resulting structure has a central double stranded “waist” from which four single stranded “arms” extend. The single-stranded arms at the ends of the monomers are designed to interact with either additional nucleic acid dendrimers or specific complementary sequences on other molecules. The arms are also designed to attach to labels. Lysine-based dendrimers and nucleic acid dendrimers are described in U.S. Pat. Nos. 5,714,166 and 5,487,973, respectively, the disclosures of which are incorporated by reference herein.

The scope of this invention also includes other types of molecular interactions such as those using antigen-antibody, receptor-hormone, receptor-ligand, agonist-antagonist, lectin-carbohydrate, Fc receptor or mouse IgG-protein A, avidin-biotin, streptavidin-biotin, amine-reactive agent-amine conjugated molecule or thiol-gold interactions, where at least one moiety is bound to a solid surface. In addition to detection, such molecular pairs may be used for purification (affinity chromatography) and separation of specific cell or phage populations during selection processes. In these instances, The presence of a higher concentration of one molecule on a solid support surface will result in a more efficient purification or separation process.

Further details regarding the practice of this invention are set forth in the following examples, which are provided for illustrative purposes only and are in no way intended to limit the invention.

### EXAMPLE I

### Affixing Dendrimeric Polyamines to the Surface of a Glass Slide Via Covalent Linkage

Attachment and hybridization efficiency of nucleic acid microarrays were enhanced using 3-aminopropyltriethoxysilane (APTES) and 1,4 phenylene diisothiocyanate (PDC) treated glass surfaces bearing polyamidoamine (PAMAM) dendrimers to increase the concentration of binding sites on the glass surface.

I. Materials and Methods:

The following protocols are provided to facilitate the practice of the present invention:

A. Preparation of Probes and Targets

Short probes and targets were commercially synthesized and purified by HPLC (Integrated DNA Technologies, Coralville, Iowa).

Oligonucleotides complementary to the FCγIIA gene were generated for use as positive controls. The first synthesized probe, FC3, was 21 bases long, amine-modified at the 3′ end and fluorescently labeled with Cy5 at the 5′ end (5′-TTT GGA TCC CAC CTT CTC CAT-3′; SEQ ID NO: 1). This probe was used as the attachment control to study the efficiency with which oligonucleotide probes attach to the glass surface. Another probe, FC2, was 21 bases long and amine-modified at the 5′ end (5′-ATG GAG AAG GTG GGA TCC AAA-3′; SEQ ID NO: 2). This probe was used as the hybridization control to study the efficiency of target molecules hybridizing to the probe molecules.

The hybridization control probe was prepared with spacer molecules (FC2SP) and without spacer molecules (FC2). Three polyethylene glycol (PEG) groups were attached to the probes as spacer molecules. Each PEG group contained 18 carbons in length and was added to the probes between the amine and the oligonucleotide moiety.

A complementary target molecule, FC3A (5′-TTT GGA TCC CAC CTT CTC CAT-3′; SEQ ID NO: 3), was also generated and 5′ end-labeled with the fluorescent dye, Cy5. This target molecule was used to passively hybridize with the FC2SP and FC2 probes.

B. Preparation of Microarray Supports

Previous experiences showed that APTES (3-aminopropyltriethoxysilane) and PDC (1,4-phenylene diisothiocyanate)-coated glass slides were appropriate for attachment of probes for heterogeneous hybridization.

Silanization (APTES coating):

150 ml of solution containing 1% (v/v) APTES (3-aminopropyltriethoxysilane) (Sigma, St. Louis, Mo.) in 95% (v/v) ethanol in water was prepared for silanizing the glass slides. After mixing the solutions, the silanization solution was titrated to pH 7.0 by adding acetic acid. A slide holding rack capable of holding twenty slides was immersed in the solution in a staining dish for twenty minutes at room temperature. Parafilm was used to seal the container to prevent the solution from absorbing moisture. After silanization, the slides were rinsed in fresh 100% ethanol at room temperature three times and then cured in a clean oven at 110° C. for twenty minutes or cured at room temperature for twenty-four hours.

1,4-Phenylene Di-isothiocynate Modification:

Silanized slides were treated with 0.2% (w/v) PDC (1,4-phenylene diisothiocyanate) (Sigma, St. Louis, Mo.) in 10% (v/v) pyridine/90% dimethylformamide (Fisher) at room temperature for two hours. The staining dish was sealed with Parafilm to prevent the solution from absorbing moisture. The slides were washed with HPLC-grade methanol and acetone, each for five minutes at room temperature and then the slides were dried in a clean oven at 110° C. for five minutes.

C. Spotting

In order to generate nucleic acid microarrays, a custom arrayer was built in the laboratory as described previously by Graves et al. (Graves, D. J., Su, H.-J., McKenzie, S. E., Surrey, S., and Fortina, P., Anal. Chem. 70 (#23), 5085-5092 (1998)). Similar arraying machines are commercially available from Biorobotics Inc., Packard Biochip Technologies LLC and GeneMachines. This moderate-cost, easy-to-build arrayer was capable of holding thirty-two 1″×3″ slides. It was also designed to hold two 96 or 384-well microtiter plates. Using this arrayer, the deposition tip was positioned with 25 μm precision. This one-tip deposition arrayer could easily generate 32 identical slides, each containing 96 or more different sample spots, and was capable of depositing spots 500 μm apart in volumes of 5 nl.

Oligonucleotide probes at concentrations of 100 μM were mixed 1:1 with Micro-Spotting solution (TeleChem International Inc, Sunnyvale, Calif.). Probes were spotted robotically by the arrayer at a volume of 5 nl and at a spacing of 500 μm from center to center. Each probe was spotted in duplicate spots in the same row in order to check the uniformity of deposition. The spotted slides were left at room temperature overnight in Petri dishes with moisture present to aid the chemical linkage of the probes to the surface.

D. Washing and Blocking

After incubating overnight to facilitate chemical linkage between the probes and glass surface, the microarray was washed to remove the unlinked probes. Spotted slides were first washed individually with 10 ml of pH 8 1×TE buffer and then washed with 10 ml of deionized water three times. The slides were then put in a 20-slide-holding-rack and washed in 55° C. deionized water for 15 minutes. After the slides were dried in a clean hood, the rack was immersed in a staining beaker with 150 ml of 1 M Tris-HCL (pH 7.5) for 1 hour. The slides were then washed individually with 10 ml of 10 M NaCl followed by 10 ml of deionized water. These steps were performed at room temperature.

E. Scanning and Data Acquisition

Fluorescently labeled arrays were scanned to quantitate the degree of hybridization. The slides were scanned using a ScanArray 5000 device (GSI Lumonics, Watertown, Mass.) or with a confocal scanner constructed in the laboratory.

II. Results:

The dendrimer treating process was as follows: After the glass slides were treated with APTES followed by PDC, a diluted solution of dendrimeric polyamine (G2, Sigma)(1 volume of dendrimer solution in 19 volumes of HPLC-grade methanol) was prepared for coating the slides with dendrimer. After treating the slides with the dendrimer solution, the slides were placed in Petri dishes sealed with Parafilm for 24 hours at room temperature. The slides were then rinsed once in methanol and dried in a clean hood. The dendrimer treating process was followed with a second PDC treating process. Then the slides were ready for spotting probes.

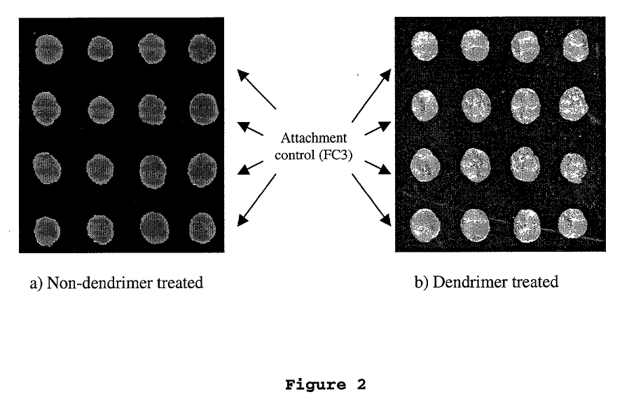

For testing attachment efficiency, the attachment control probe, FC3, was spotted onto the surface of the dendrimeric polyamine-bearing nucleic acid microarray in a volume of 5 nl and at a concentration of 5 μM in Micro-Spotting solution (Telechem International, Inc.). Both dendrimer treated and non-dendrimer treated slides were spotted, and each slide contained 16 duplicate spots in 4 rows of 4 spots each (FIG. 2). The nucleic acid microarrays were then incubated overnight at room temperature with moisture present to complete the reaction. Any unbound probe was washed away. The slides were scanned at the same combination of photo-multiplier voltage (PMT) and laser power on a GSI 5000 scanner. The PMT varied between 750-1000 volts depending on the particular samples scanned.

In addition to the attachment control probe, a hybridization control probe, FC2, was also used. FC2 hybridized specifically to a very short (20 base) dye-labeled target, FC3A, and was used to compare the signals on individual glass slides. Since this hybridization control probe/target pair hybridized very easily and consistently, it was used for slide comparisons to avoid discrepancies due to differing reaction conditions and different PMT and laser power settings. This step also eliminated the need for data normalization when PMT and laser power settings differed.

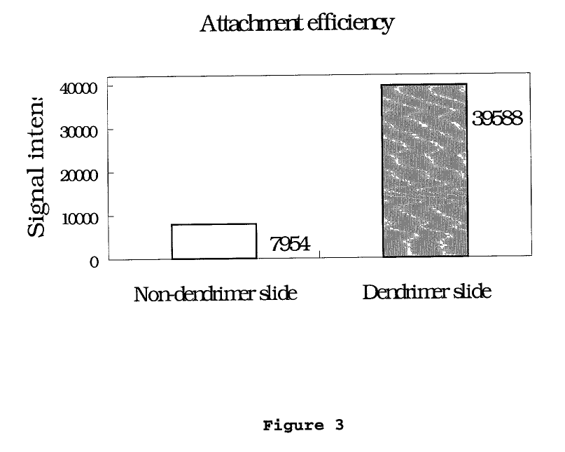

The scanned images of the fluorescence measured from the microarrays are shown in FIG. 2 and the signal intensities measured from these arrays are provided in FIG. 3. The results indicate that the signal from the fluorescently-labeled microspots on the dendrimeric polyamine-bearing glass slide was approximately 5 times (39588/7954) greater than the non-dendrimer-treated slide. Thus, the addition of dendrimers increased substantially the number of probes bound to the glass surface.

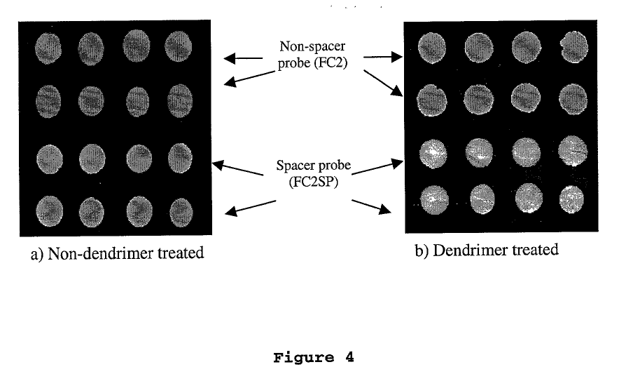

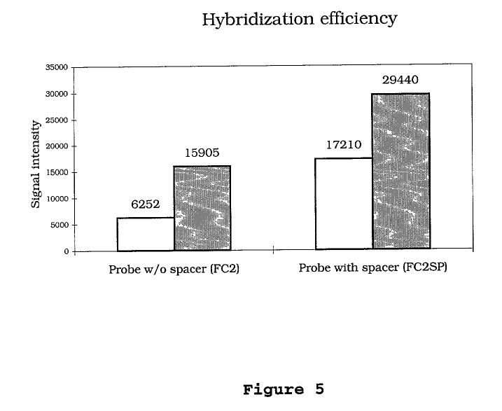

To test the hybridization efficiency as well as the possibility of using dendrimers to replace the spacer molecules, a spacer hybridization control oligonucleotide probe, FC2SP, was generated. Compared to the hybridization control probe, FC2, the spacer probe, FC2SP, was an identical molecule except for the addition of three spacer groups between the amine and the oligonucleotide. The FC2 and FC2SP probes were spotted in volumes of 5 nl at concentrations of 50 μM in Micro-Spotting solution on both the dendrimeric polyamine-bearing and non-dendrimer-treated slides. Each slide was spotted with both probes, 8 duplicates in 2 rows of 4 spots. The slides were incubated overnight and washed, followed by blocking before they were both passively hybridized at 48° C. for 5 minutes with 25 μl of hybridization solution (6×SSPE+1 mM CTAB) containing 100 fmole of perfectly matched target molecule, FC3A. Following the washing step, the slides were scanned at the same combination of PMT voltage and laser power as described previously and their signal intensities were compared. The scanned images of the fluorescence measured form these microarrays are shown in FIG. 4 and the signal intensities measured from these arrays are provided in FIG. 5.

It was found that the use of the dendrimeric polyamine-bearing glass slide resulted in a higher hybridization efficiency regardless of whether a spacer was used or not. In other words, the dendrimeric polyamine-bearing slide increased the hybridization efficiency by a factor of 1.71 (29440/17210) for a spacer-containing probe and 2.54 (15905/6252) for a non-spacer-containing probe, relative to the non-dendrimer-treated slide. The signal from the spacer probe (17210) on the non-dendrimer-treated slide was also very close to the signal from non-spacer probe (15905) on the dendrimer-treated slide. This suggested that similar hybridization results are obtainable by substituting dendrimer for spacer groups.

These results demonstrate that the addition of dendrimers to APTES/PDC-treated glass slides increased binding of the amino-modified probes and also caused an incremental change in hybridization efficiency. In addition, the relatively low-cost dendrimer solution was found to effectively replace the high-cost spacer groups used in oligonucleotide probe synthesis. Thus, dendrimeric polyamine-bearing microarray slides may be used to advantage by those skilled in the art to improve upon existing microarray analyses by increasing the sensitivity and efficiency of hybridization reactions using nucleic acid microarrays.

### EXAMPLE II

### Affixing Dendrimeric Polyamines to the Surface of a Glass Slide Via Physical Adsorption

Polyamidoamine (PAMAM) dendrimers were physically adsorbed to the surface of glass slides and used to immobilize oligonucleotides in order to improve the attachment and hybridization efficiency of nucleic acid microarrays.

I. Materials and Methods:

The following protocols are provided to facilitate the practice of the present invention:

A. Pretreatment of Glass Slides:

Corning glass slides were pre-cleaned using 1 M potassium hydroxide at 75° C. for 20 minutes in order to increase the net negative charge on the glass surface.

B. Preparation of Dendrimeric Polyamine Solutions and Attachment of Dendrimers to Glass Surface:

Dendrimer solutions of G2, G3 and G4 PAMAM dendrimers were produced according to the specifications provided in Table 1:

TABLE 1
DENDRIMER SOLUTIONS

| | Number of | | | |
|---|---|---|---|---|
| | Terminal | Initial | | Final |
| Dendrimer | Amine | Concentration | | Concentration |
| Generation | Groups | (wt %) | Dilutions | (wt %) |
| G2 | 16 | 20 | 50:1, | 0.4, 0.2, |
| | | | 100:1, | 0.1, 0.02, |
| | | | 200:1, | 0.004, |
| | | | 1000:1, | respectively |
| | | | 5000:1 | |
| G3 | 32 | 20 | 100:1, | 0.2, 0.1, |
| | | | 200:1, | 0.02, 0.004, |
| | | | 1000:1, | respectively |
| | | | 5000:1 | |
| G4 | 64 | 10 | 50:1, | 0.1, 0.05, |
| | | | 100:1, | 0.01, 0.002, |
| | | | 500:1, | respectively |
| | | | 2500:1 | |

A total of thirteen dendrimer solutions were prepared at various concentrations for each dendrimer generation and placed in 20 ml plastic slide containers. Five glass slides were inserted into each container, and the containers were sealed and wrapped with parafilm to prevent evaporation. All of the slides were incubated in their respective solutions for 16 hours. The slides were then removed from their containers and dried under ambient conditions in biological safety cabinets to eliminate dust or other particles from contaminating the treated slides. The slides were handled at all times wearing polyethylene gloves and a Teflon coated tweezer to further eliminate surface contamination.

The dendrimeric polyamine-bearing glass slides were then stored under ambient conditions for a period of two weeks after deposition. This hiatus was utilized because previous studies with polylysine slides indicated improved stability of the treated slides when aged (data not shown).

C. DNA Microarray Hybridization Reactions:

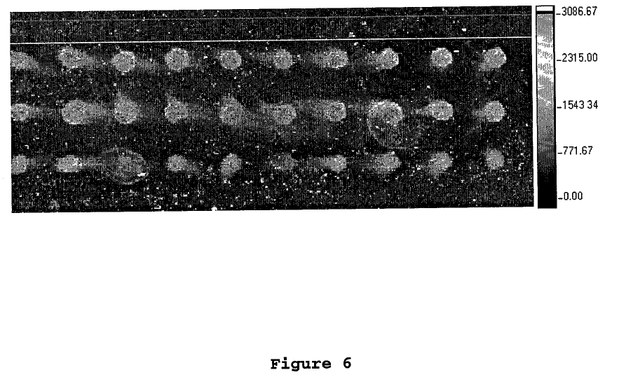

Two dendrimeric polyamine-bearing glass slides of each generation and concentration were spotted with DNA oligonucleotide probes using a robotic pipettor constructed in the laboratory. Two double-stranded oligonucleotide probes, 300 and 600 base pairs (bp) in length, were micro-spotted onto the surface of the glass slides. DNA concentrations were measured using a Hoeffer mini-fluorometer and Hoescht 33258 dye. The concentration of the 300 and 600 bp DNA molecules were 160 ng/ml and 140 ng/ml, respectively. Both oligonucleotide probes were prepared with ArrayIt® microspotting solution (TeleChem International, Sunnyvale, Calif.) in a 2:1 ratio, DNA:ArrayIt®. The probe solutions were then arrayed onto the glass surface. Each spot contained 10 nl of DNA solution and the spots were spaced 1000 microns apart (FIG. 6). The spotted slides were then stored in slide boxes under ambient conditions for three days prior to labeling with the fluorescent dye, YoYo-1 (Molecular Probes, Eugene, Oreg.).

YoYo-1 dye was diluted 4000:1 with HPLC water to a final concentration of 0.25 μM. The dye was shielded from light at all times. In order to cover the spotted array completely, 250 μl of dye was pipetted onto the slides. After 5 minutes, the dye was rinsed off of the slides using 10% 10×TAE buffer (Life Technologies, Rockville, Md.).

D. Analysis of Spotted Microarrays:

The spotted slides were analyzed using a laser-scanning microarray analysis system constructed in the laboratory. Similar laser-scanning systems are commercially available from Packard Biochip Technologies LLC. This system measured the fluorescent signal intensity from each spot within a relatively short time period depending on the overall size of the array (i.e., the number of spots and their spacing). The signal intensity of the spots' background was also measured to provide a signal to noise (S/N) ratio for each spot, row or the entire array.

II. Results:

A. Relationship Between DNA Adsorption and Dendrimer Concentration:

To determine the relationship between dendrimer generation and the level of DNA adsorption to the glass slide, G2, G3 and G4 dendrimeric polyamine solutions were prepared at various concentrations and affixed onto glass slides (See Table 1). Two oligonucleotide probes, 300 bp and 600 bp, respectively, were then micro-spotted onto the dendrimeric polyamine-bearing glass slides. FIG. 6 provides a qualitative contrast between three probe solutions spotted on a dendrimeric polyamine-bearing glass slide. This particular slide was treated with 0.1 wt % G2 PAMAM dendrimer. The top row was spotted with the 600 bp oligonucleotide probe in ArrayIt® solution and the middle row was spotted with the 300 bp oligonucleotide probe in ArrayIt® solution. The bottom row was spotted with the 300 bp oligonucleotide probe prepared without the ArrayIt® solution. The results obtained show that the spotted oligonucleotide/ArrayIt® solutions produced well-defined spots on the dendrimer-bearing slides.

The relative amount of DNA adsorbed as a function of dendrimer concentration and generation was measured and the results are provided in Table 2. The spot intensity values represent the level of fluorescence measured from each microspot.

TABLE 2
DNA SIGNAL INTENSITY VS. DENDRIMER
CONCENTRATION & GENERATION

| Dendrimer | | | Avg. Spot | | |
|---|---|---|---|---|---|
| Generation | Dilution | wt % conc. | Intensity | Background | S/N |
| G2 | 50:1 | 0.4 | 11336 | 1610 | 7.04 |
| | 100:1 | 0.2 | 4844 | 823 | 5.89 |
| | 200:1 | 0.1 | 4481 | 1126 | 3.98 |
| | 1000:1 | 0.02 | 2996 | 945 | 3.17 |
| | 5000:1 | 0.004 | 2503 | 1132 | 2.21 |
| G3 | N/A | N/A | N/A | N/A | N/A |
| G4 | 50:1 | 0.2 | 2375 | 645 | 3.68 |
| | 100:1 | 0.1 | 2800 | 586 | 4.78 |
| | 500:1 | 0.02 | 1587 | 397 | 4.00 |
| | 2500:1 | 0.004 | 927 | 396 | 2.34 |

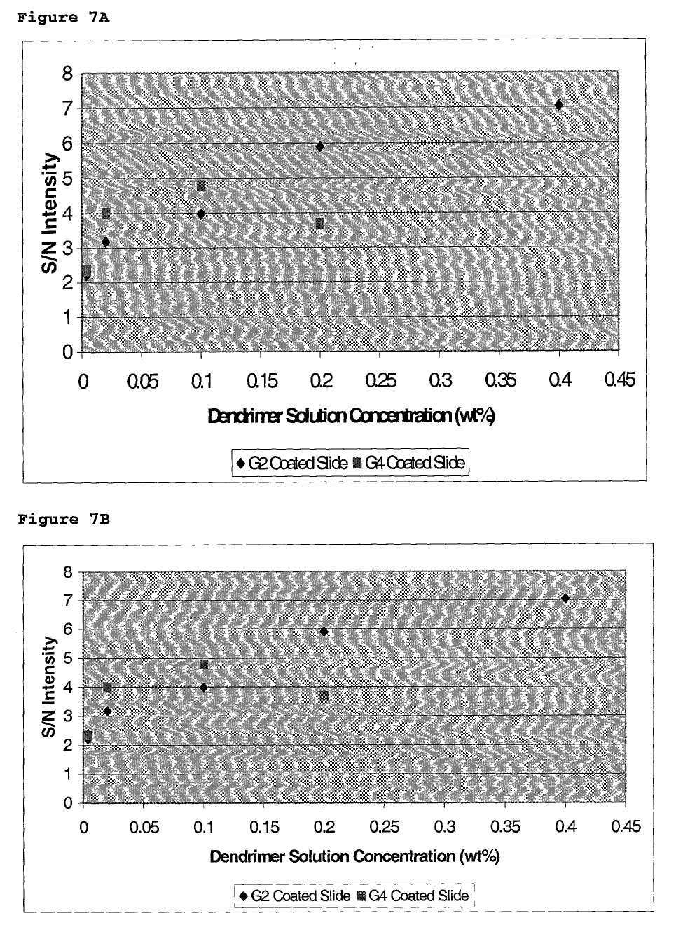

The slides treated with G3 PAMAM dendrimers were unable to produce consistent or measurable results. This result suggested that the material supplied by the manufacturer was defective. The results presented in Table 2 are further illustrated in FIGS. 7A and 7B.

B. Relationship Between Dendrimer Adsorption Time and Nucleotide Binding:

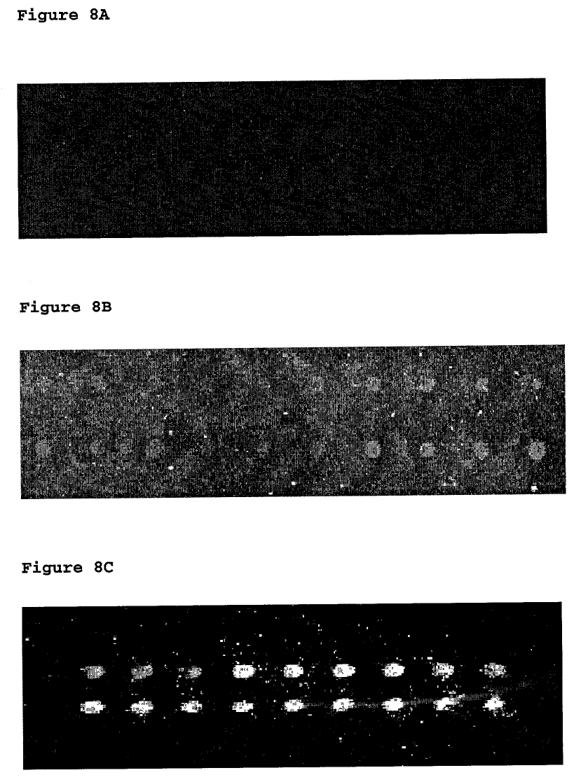

Results from previous experiments (data not shown) demonstrated that glass slides coated with polylysine required a two (2) week delay between the adsorption of an organic compound on silica and DNA spotting to allow for surface diffusion, bond re-arrangement, or a combination thereof of the amino-containing molecules. Otherwise, the nucleic acids did not bind well enough to the glass surface to be utilized as probes. To determine if a similar incubation period was required for the dendrimeric polyamine-bearing glass slides, three (0.2 wt %) G2 PAMAM dendrimer slides were spotted with the 600 bp oligonucleotide probe at 1 day, 1 week, and 2 weeks after dendrimer deposition. FIGS. 8A-8C show the relationship between the time delay dendrimer treatment to oligonucleotide spotting and the attachment of measurable microarray spots on the glass slides. The quantitative measurements are also provided in Table 3.

TABLE 3
DELAY EFFECT BETWEEN DENDRIMER DEPOSITION
AND DNA SPOTTING

| Time | Avg. Spot | Background | |
|---|---|---|---|
| Delay | Intensity | Intensity | S/N |
| 1 Day | N/A - Washout | 302 | N/A |
| 7 Days | 1355 | 481 | 2.8 |
| 15 Days | 9820 | 365 | 24.8 |

These results indicate that the optimal use of the dendrimeric polyamine-bearing glass slides occurs approximately two weeks after the dendrimers are adsorbed to the slides.

C. The Effect of Dendrimer pH on DNA Binding:

The effect of dendrimer pH on DNA binding was also investigated. Two additional 0.1 wt % G2 PAMAM dendrimer solutions were prepared at pH 6.7 and pH 11.5, respectively. The normal pH of the PAMAM dendrimer solutions was approximately 9.5. Glass slides were treated with the respective dendrimeric polyamine solutions for 16 hours. After two weeks, the slides were spotted with the 600 bp oligonucleotide probe and analyzed. The results of the dendrimer pH experiments are provided in Table 4.

TABLE 4
DENDRIMER PH EFFECT ON DNA BINDING

| Dendrimer pH | Spot Intensity | Background | S/N |
|---|---|---|---|
| 6.7 | Washout | 22 | N/A |
| 6.7 | Washout | 64 | N/A |
| 9.5 | 4481 | 1126 | 3.98 |
| 11.5 | 458 | 367 | 1.25 |
| 11.5 | 433 | 386 | 1.12 |

The results of these pH experiments reinforce the need for dendrimer deposition to occur at a pH conducive to maximizing the charge interaction between silica and the dendrimers' terminal amino groups. The charge interaction was reduced at pH 6.7, and the low signal intensities were probably the result of limited dendrimer adsorption. Although the higher pH should have increased the charge interaction, silica dissolves readily above pH 9.8 and this dissolution may have competed with the adsorption process.

III. Discussion:

The S/N ratios in all of the experiments described above provided a measure of the dendrimeric polyamine-bearing slides' capabilities to adhere DNA to a desired location. In the dendrimer concentration and generation experiments, the S/N ratios for G2 PAMAM dendrimers increased steadily as the G2 PAMAM dendrimer concentrations increased (FIG. 7B), whereas the G4 dendrimers, molecules that are 4 times the size of the G2 dendrimers, had an apparent leveling off in their ability to anchor DNA somewhere between 0.1 and 0.2 wt % deposition concentration (FIG. 7A). This leveling off phenomenon was expected to take place at some dendrimer concentration, since the dendrimers will eventually coat the entire surface of the glass slide and provide a uniform positively charged surface for the DNA to bind to. However, it was surprising that such a high dendrimer concentration was necessary for this to occur. Theoretically, a 0.01 wt % concentration of dendrimer would be more than sufficient to coat the entire slide. Whether or not higher dendrimer concentrations are necessary to help drive dendrimer adsorption or whether the topography created by the dendrimers is responsible for this phenomenon remains to be determined.

Based on the foregoing results, the use of dendrimeric polyamines to enhance nucleic acid microarray hybridization reactions was successfully demonstrated. This method improves substantially the surface molecule density and hybridization efficiency on nucleic acid microarrays.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for preparing microarrays of biological entities on a solid support, said biological entities having at least one amine reactive functional group, said method comprising the steps of:
a) treating said solid support to affix preformed dendrimeric polyamine to a surface of said solid support; and
b) microspotting biological entity-containing solution on said treated solid support under conditions effecting chemical binding of biological entities to the amine groups of said dendrimeric polyamines thereby providing a microarray of said biological entities on said solid support.

2. The method of claim 1, wherein said biological entities are selected from the group consisting of nucleic acid molecules, peptides, phages, eucaryotic cells, procaryotic cells and viruses.

3. The method of claim 1, wherein said biological entities are nucleic acid molecules.

4. The method of claim 1, wherein said dendrimeric polyamine is affixed to said solid support via physical adsorption.

5. The method of claim 1, wherein said dendrimeric polyamine is affixed to said solid support via covalent bonding.

6. The method of claim 1, wherein said dendrimeric polyamine is selected from the group consisting of symmetrical and unsymmetrical branching dendrimers, polyamidoamine (PAMAM) dendrimers, lysine-based dendrimers and nucleic acid dendrimers.

7. The method of claim 1, wherein said dendrimeric polyamines are PAMAM dendrimers.

8. The method of claim 1, wherein said solid support is a glass slide.

9. The method of claim 4, wherein said treated solid support is stored for a predetermined time period before carrying out the microspotting step.

10. The method of claim 9, wherein said predetermined time period is at least two weeks.

11. A nucleic acid microarray comprising a solid support to which is affixed preformed dendrimeric polyamine, and a plurality of different nucleic acid probes chemically bound to said dendrimeric polyamine, said chemically bound nucleic acid probes forming a pattern of discrete microspots on said solid support, each of said microspots being composed of a different one of said nucleic acid probes.

12. A method of analyzing a test sample comprising at least one target nucleic acid molecule, comprising the steps of:
a) providing a nucleic acid microarray according to claim 1;
b) contacting said test sample with said nucleic acid microarray under conditions causing the formation of hybrids between said target nucleic acid molecule and said nucleic acid probes on said nucleic acid microarray;
c) labeling with a detectable reporter substance one member selected from the group consisting of said target nucleic acid molecule and any hybrids form in step b; and
d) detecting the occurrence of said detectable reporter substance in said nucleic acid microarray.

13. The method of claim 12, wherein said detectable reporter substance is selected from the group consisting of chemiluminescent, enzymatic, radioactive and fluorescent materials.

14. The method of claim 12, wherein said detectable reporter substance is fluorescent material.
